(19) United States
(12) Patent Application Publication
(10) Pub. No.: US 2009/0131521 A1
(43) Pub. Date: May 21, 2009

(54) Aqueous retinoid and benzoyl peroxide gel

(76) Inventors: Mitchell S. Wortzman, Scottsdale, AZ (US); Waranush Jitpraphai, Chandler, AZ (US); Manzer Durrani, Scottsdale, AZ (US)

Correspondence Address: REED SMITH LLP, 2500 ONE LIBERTY PLACE, 1650 MARKET STREET, PHILADELPHIA, PA, 19103, US

(21) Appl. No.: 12/288,411

(22) Filed: Oct. 20, 2008

Related U.S. Application Data
(60) Provisional application No. 60/999,620, filed on Oct. 18, 2007.
(60) Provisional application No. 61/046,308, filed on Apr. 18, 2008.

(51) Int. Cl. A61K 31/327; A61K 31/19; A61P 17/10

(52) U.S. Cl. 514/557; 514/714

(57) ABSTRACT

Embodiments of this invention relate to a composition containing both benzoyl peroxide and a retinoid. Additionally, it relates to the treatment of acne vulgaris by applying an aqueous gel comprising BPO and a retinoid.

### RELATED APPLICATIONS

This application claims the benefit of U.S. Provisional Application No. 60/999,620, filed Oct. 18, 2007, and U.S. Provisional Application No. 61/046,308, filed Apr. 18, 2008.

### FIELD OF THE INVENTION

This invention relates to a composition containing both benzoyl peroxide (“BPO”) and a retinoid, preferably tretinoin. Additionally, it relates to the treatment of acne vulgaris, referred to as acne, by either simultaneously applying benzoyl peroxide and a retinoid, preferably tretinoin, in an aqueous gel or sequentially applying an aqueous gel with benzoyl peroxide and an aqueous gel with a retinoid, preferably tretinoin. A novel method of including tretinoin and benzoyl peroxide in a unique drug delivery system is disclosed where benzoyl peroxide and tretinoin are incorporated in a steric stabilized emulsion complex formulated with hydrophobically modified carbomers; which ensures stability of the two incompatible active ingredients.

### BACKGROUND

Acne is a disease of the skin in which the pilosebaceous structures of the skin become inflamed, leading to the formation of comedones, pustules and nodules. Acne can lead to permanent scarring in severe cases.

It is generally believed that acne arises when hyperkeratosis of the pilosebaceous structure wholly or partially blocks the opening of the structure, resulting in comedones filled with sebum, keratin, and Propionibacterium acnes (“P. acnes”). These lesions are commonly identified as acne. P. acnes naturally occurs in normal skin, but is especially and characteristically present in acne lesions. It is believed that metabolic byproducts and waste from P. acnes within the pilosebaceous structures cause or contribute to the inflammation of acne lesions.

Conventional acne treatments have taken many forms. Oral drugs including tetracycline, minocycline, doxycycline, and erythromycin. Topical keratolytic agents, such as salicylic acid are sometimes used. Keratolytic agents are thought to encourage the opening up of blocked pilosebaceous structures, thereby reducing conditions that are favorable to inflammation. Benzoyl peroxide, an anti-microbial, remains a popular and effective treatment. Topical antibiotics, such as clindamycin, which are effective against P. acnes, have also been used with a view towards preventing the formation of metabolic byproducts from this organism. Topical retinoids such as tretinoin have also been used in the treatment of acne.

In this specification, the term “retinoids” means structural retinoids such as retinol, retinal, tretinoin (all-trans retinoic acid), retinoic acid, isotretinoin, alitretinoin, as well as functional retinoids which bind to retinoid receptors, such as adapalene and tazarotene, and mixtures thereof, unless otherwise stated. In this specification, “aqueous gel” means a gel comprising water and no alcohol, unless otherwise stated.

Tretinoin and other retinoids have been extensively used as an acne treatment based upon their ability to reverse abnormal desquamation and suppression of toll-like receptor 2(TLR-2). It was known as a treatment that was likely to irritate the skin. Bazzano (U.S. Pat. No. 5,721,275, herein incorporated by reference) advanced tretinoin treatment by inventing a stable, minimally irritating aqueous gel composition which contained tretinoin. The stability of the tretinoin in aqueous gel was significantly better than tretinoin in a cream and tretinoin in an alcoholic gel (such as Retin-A gel). Bazzano's composition appeared to allow for the slow release of tretinoin into the skin, which caused the patient to experience much less irritation.

Benzoyl peroxide (C₁₄H₁₀O₄) also treats acne due to its ability to suppress P. acnes. benzoyl peroxide is insoluble in water, therefore the prior art compositions contain benzoyl peroxide in suspension. One example of a benzoyl peroxide alcoholic gel in the prior art is Medicis' Triaz Gel (3%, 6%, and 9%). Bazzano taught nothing about combining tretinoin with benzoyl peroxide for acne treatment.

Physicians have long tried to combine two or more medications, which each treat a disease or condition, for better results. However, when two medications are incompatible or the combination of the medications causes at least one of them to degrade, the combination treatment is no longer as desirable or useful.

In the prior art, it was thought that benzoyl peroxide and retinoids, especially tretinoin could not be used in a combination formulation. As Cotterill said in “Benzoyl Peroxide,” Acta Dermatology (Stockholm), Suppl. 89, 1980 (herein incorporated by reference), “retinoic acid and benzoyl peroxide are chemically incompatible, as the latter will oxidize the former.” Further, the prior art noted that if benzoyl peroxide and tretinoin were both used in acne treatment, that it should be in sequence with 8-12 hours between applications. For example, benzoyl peroxide would be used in the morning and tretinoin at night.

### SUMMARY OF THE INVENTION

One embodiment of the present invention is a composition comprising a semi aqueous gel composition including water, benzoyl peroxide, a retinoid, and a polymeric gelling agent. In certain embodiments, the polymeric gelling agent includes an emulsifying polymer.

Another embodiment includes a method of treating acne comprising applying to skin an aqueous gel comprising benzoyl peroxide and a retinoid.

Still another embodiment includes a composition comprising tretinoin at about 0.025 weight %, benzoyl peroxide at about 5 weight %, a polymeric gelling agent, and water, wherein the composition is substantially free of alcohol.

### DETAILED DESCRIPTION

The present invention is a significant departure from the generally accepted wisdom in the prior art. The prior art taught that if retinoids, e.g. tretinoin, and benzoyl peroxide were applied to the skin in sequence without at least 8 hours in between, the retinoid would be oxidized (and therefore degraded and not useful for acne treatment).

Bazzano reports that aqueous gels are very useful in forming stable tretinoin compositions. Medicis' Ziana® gel is a commercial embodiment of such a stable tretinoin aqueous gel with a low level of irritation. Ziana® gel contains 1.2% clindamycin phosphate and 0.025% tretinoin, some of which is in solubilized form and other in suspended crystalline form. Ziana® gel also includes purified water USP, glycerin USP, carbomer 981 NF, methylparaben NF, polysorbate 80 NF, edetate disodium USP, citric acid USP, propylparaben NF, butylated hydroxytoluene NF and tromethamine USP. In this specification (including the claims), % means weight percent of the total composition unless otherwise stated.

Without wishing to be limited to a particular mechanism, it is believed that the stability of the tretinoin is due to solubilizing the tretinoin and slowly releasing it over time. It is believed that the solubilized form of tretinoin will largely penetrate the skin before the crystalline tretinoin. The crystalline tretinoin is less likely to immediately release into the skin upon application, because it must first be solubilized on the skin.

The invention takes this slowly releasing and stable retinoid, preferably tretinoin aqueous gel and incorporates benzoyl peroxide. Without being limited to mechanism, it is believed that the benzoyl peroxide is separated from the solubilized tretinoin in the aqueous gel, the crystalline tretinoin, is immediately released into the skin and therefore there is no long exposure of the tretinoin to the benzoyl peroxide and the expected oxidation does not occur.

One embodiment of the present invention is a composition containing both a retinoid, preferably tretinoin and benzoyl peroxide in an aqueous gel, wherein the benzoyl peroxide does not oxidize the tretinoin or oxidizes at a rate slow enough to allow the composition to remain pharmaceutically active during the residence time on human skin.

### Composition

One embodiment of the invention is a composition which comprises retinoid, e.g., tretinoin, and benzoyl peroxide in an aqueous gel, which does not contain alcohol. It is preferable that at least a portion of the tretinoin is solubilized, more preferable that at least 50% of the tretinoin is solubilized. The preferred particle size of the crystalline tretinoin is at least about 50% of particles ≦about 10 μm in size and at least about 90% of particles ≦about 20 μm in size. It is preferable that the composition comprises at least 40% to 50% water, and usually more.

In another embodiment, tretinoin is present at about 1.0% to about 0.01%, preferably about 0.025%, and benzoyl peroxide is present at about 3% to about 9%, preferably about 5%.

In another embodiment, an aqueous gel comprising a retinoid, preferably tretinoin, is applied to the skin and within a short time (either before or after), a benzoyl peroxide composition, which may be a gel, preferably aqueous gel, is applied to the skin. The application of the retinoid aqueous gel and the benzoyl peroxide gel may be in any sequence, preferably with the retinoid aqueous gel first.

The gelling agents useful in the present invention are those which form a gel in aqueous medium and hold the retinoid for slow release and maintaining the integrity of the retinoid. The gel may be formed by any known gelling agent, including but not limited to, polymeric gelling agents, including high molecular weight copolymers of polyacrylic acids, for example, Carbopol®, (CAS 9003-01-4), and related polymers which are known agents for use in various types of pharmaceutical and cosmetic compositions. The crosslinked polymer of polyacrylic acids swells to form a gel when neutralized with a base, such as sodium hydroxide or an amine, to a pH above about 4 to about 6.

In embodiments of this invention, the gel can be a gelled aqueous phase of an oil-in-water emulsion. This can be accomplished through the use of a gel forming, polymeric emulsifier, such as gel forming polyacrylic or poly alkyl acrylate polymer emulsifiers such as Pemulen® TR-1, a Pemulen® TR-2, Pemulen® TR-1 NF, or Pemulen® TR-2 NF, which are emulsifying high molecular weight acrylic acid/C10-C30 alkyl acrylate copolymers.

In some embodiments, a topical gel formulation comprising both benzoyl peroxide, and tretinoin is an oil-in-water emulsion, where the aqueous phase is gelled. This can be accomplished by use of emulsifying acrylic acid/C10-C30 alkyl acrylate copolymer. For example, in one embodiment, the surfactant/emulsifying agent may be commercially available under the trademark Pemulen® TR-1 and Pemulen® TR-2. In other embodiments, the surfactant or emulsifier may be Pemulen® TR1 NF and/or Pemulen® 11 NF. Pemulen® polymeric emulsifiers are predominantly high molecular weight polyacrylic acid polymers. These novel primary emulsifiers have a small oil-loving (lipophilic) portion in addition to a large, water-loving (hydrophilic) portion. This chemical structure allows these copolymers to function as primary emulsifiers in oil-in-water emulsions. Whereas Carbopol® water soluble polymers have proven useful as secondary oil-in-water (o/w) emulsion stabilizers, Pemulen® polymers can actually form o/w emulsions. The lipophilic portion adsorbs at the oil-water interface, and the hydrophilic portion swells in the water forming a gel network around oil droplets to provide exceptional emulsion stability to a broad range of oils.

In preferred embodiments, the topical gel formulation comprises about 0.1% to about 1% by weight of emulsifying surfactant and about 4% to about 80% by weight of water. In some embodiments, the topical gel formulation further comprises about 1% to about 5% by weight of hydrophilic polymer and about 15% to 30% by weight of fatty base. Examples of suitable hydrophilic polymers include, but are not limited to, cellulose derivatives such as hydroxypropyl cellulose (HPC), hydroxypropyl methylcellulose (HPMC), hydroxyethyl cellulose, and ethylcellulose. The use of hydroxypropyl methylcellulose is particularly preferred in some embodiments. In some embodiments, the fatty base may be selected from C12-C18 fatty acids or the esters thereof, silicon, Vaseline or paraffin oils.

Additional ingredients in the composition may include, without limitation, viscosity adjusters, propellants, odor modifiers, surfactants, fragrances, antioxidants, colorants, preservatives, emulsifiers, and pH adjusters.

Additionally, other additional active ingredients may be included in the composition, such as, for example, antimicrobial agents, antiinflammatory agents, keratinization modulators, depigmenting agents, immunomodulators, antifungals, and analgesics.

Compositions according to this invention may also include both natural and semi-synthetic antibiotics effective against P. Acnes. The tetracycline class of antibiotics is useful in this regard. Examples include tetracycline, doxycycline, and minocycline. Lincosamide antibiotics are also useful in this regard. Examples include clindamycin and lincomycin. Antibiotics effetive agains P. Acnes can be used in any acceptable form, such as salts and esters. Examples include clindamycin hydrochloride, clindamycin palmitate, clindamycin phosphate, minocycline hydrochloride and other such compounds.

Other antimicrobial agents useful in the present embodiments of the invention include, such as, without limitation, povidone iodine, hexachlorphene, sulfasalazine, sulfasoxazole, acetylsulfasoxazole and combinations thereof.

Antiinflammatory agents may include, without limitation, aldometasone, amcenonide, betamethasone, esters of betamethasone, desonide, clobetasole propionate, clocortolone pivilate, triamcinilone acetonide, desoximetasone, diflorosone, mometasone furoate, prednicarbate, cluocinonide, fluocinolone acetonide, hydrocortisone and combinations thereof. In one embodiment, the composition further comprises clindamycin.

Compositions according to the present disclosure may also advantageously include antioxidants. These compounds may act to stabilize the compositions, exert an antioxidant effect on the skin, or may perform both functions. Examples of antioxidants useful in this respect are ascorbic acid, ascorbyl fatty acid esters, vitamin E, vitamin E derivatives such as tocopheryl phosphate, alpha lipoic acid, epicatechins, BHT, and isoflavones.

Keratinization modulators may include, without limitation, retinoids, alpha hydroxy acids, beta hydroxy acids, salicylic acid, resorcinol, and combinations thereof.

Immunomodulators may include, without limitation, cylclosporine, imiquimod, fluorouracil, podophilox, podophyllin, and combinations thereof.

Antifungal agents may include, without limitation, nystatin, ciclopirox and ciclopirox olamine, griseofulvin, itraconazole, fluconazole, ketoconazole, terbinafine, econazole, benzyl alcohol, undecylenic acid and salts thereof, benzyl benzoate and combinations thereof.

### EXAMPLE 1

The degradation profile of tretinoin in Ziana® gel when mixed with equal volume benzoyl peroxide 6% gel over a 24 hour period (without UV light exposure) after incubation at 35° C. was measured. Tretinoin assays were obtained at baseline, 2, 4, 6, 8 and 24 hours.

TABLE 1

| Time (hours after baseline) | % Potency of Tretinoin |
|---|---|
| 0 | 101.6 |
| 2 | 101.6 |
| 4 | 99.2 |
| 6 | 98.4 |
| 8 | 96.8 |
| 24 | 79.8 |

The tretinoin remained stable with a 96.8% potency after 8 hours, suggesting that the application of the benzoyl peroxide aqueous gel and tretinoin aqueous gel together (or in close sequence) does not cause rapid degradation of the tretinoin.

### EXAMPLE 2

The degradation and penetration of tretinoin in Ziana® gel was studied with and without application of 5% benzoyl peroxide gel and with and without UV exposure.

Human cadaver trunk skin was mounted onto 1.0 cm Franz Diffusion Cells, and washed. The reservoir solution was phosphate-buffered isotonic saline (pH 7.4+−0.01). Separate chambers were set up to house the cells. One set of chambers was dosed and sampled under control non-UV light exposure. When not being sampled, the chambers were maintained in the dark. The other set of chambers had the skin surface exposed to a solar simulator light source (KBD Custom Research, Inc.'s FS24772 UVB-HO) at 33 inches above the skin surface. The UV light was provided at 20 minute durations following each dose application and sample collection.

A dose of 5 μL formulation/cm² of Ziana® gel was applied to the outer surface of the skin. After 2 hours, 5 μL formulation/cm² of benzoyl peroxide 5% gel was applied to the outer surface of the skin. Tretinoin and isotretinoin drug absorption was measured by monitoring its rate of appearance in the reservoir solution bathing the inner surface of the skin. Isotretinoin is a degradation product of tretinoin. Following dose application, the surface of selected chambers were washed twice (0.5 mL each) with 80% isopropanol and 20% water to collect formulation from the surface of the skin. Following the wash, skin surface was tape stripped to collect stratum corneum. Following tape stripping, the skin was removed from the chamber, and split into epidermis and dermis. All skin layers were extracted overnight in 80% isopropanol and 20% water.

For the glass dish samples, about 20 g Ziana® gel was prepared and in separate dishes, a mixture of Ziana® gel (20 g) and benzoyl peroxide 5% gel (20 g) was prepared and well-mixed. At 2, 4, 6, and 8 hours, following re-mixing of the formulations, three 100 μL aliquots of formulation were collected from each dish, mixed with 80:20 isopropanol:water and saved for subsequent analysis.

All samples were processed and analyzed for tretinoin and its active isomer, isotretinoin. The degradation of tretinoin in Ziana® gel was evaluated over 24 hours at 32.0+−1.0° C. The assays were evaluated at baseline, 2, 4, 6, 8 and 24 hours after baseline.

The tretinoin concentrations were analyzed by high performance liquid chromatography (“HPLC”). A Hewlett-Packard 1100 Series HPLC system was used with an Agilent 1100 Series LC with a diode array detector. A solvent system consisting of 5% 0.1M ammonium acetate (pH 5.0) with acetic acid and 95%/5% acetic acid/acetonitrile was run through a Phenomenex Luna C18(2)-100 A column (100×4.6; 3μ) at a flow rate of 0.5 mL/min. Ten microliters of sample were injected.

The data over a 24 hour period shows that tretinoin from Ziana® gel does penetrate human skin; it starts slow and has a progressive rise in epidermal and dermal concentrations.

When the skin is exposed to benzoyl peroxide aqueous gel 2 hours after the Ziana® gel application, there was no appreciable effect on tretinoin penetration under light conditions. A slight increase in tretinoin penetration appeared to occur after benzoyl peroxide exposure under dark conditions between 4 and 12 hours after the Ziana® gel application.

### EXAMPLE 3

### Total Skin Content for Tretinoin Results Across Donors

Percutaneous absorption of tretinoin with and without benzoyl peroxide into Human Cadaver Skin under light and dark conditions from a single application (Mean±SE (n=2) as Percent of Applied Dose)

TABLE 2

| | Ziana ® | | | |
|---|---|---|---|---|
| | Gel with | | | |
| | benzoyl | | Ziana ® Gel | Ziana ® Gel |
| | peroxide | Ziana ® Gel | without | without |
| | Gel | with benzoyl | benzoyl | benzoyl |
| Exposure | Skin | peroxide Gel | peroxide Gel | peroxide Gel |
| Duration | Content | Skin Content | Skin Content | Skin Content |
| (hours) | % (Light) | % (Dark) | % (Light) | % (Dark) |
| 2 | 0 | 0.220 ± 0.220 | 0 | 1.164 ± 0.344 |
| 4 | 0 | 1.018 ± 1.018 | 0 | 0.327 ± 0.327 |
| 6 | 0 | 0.385 ± 0.385 | 0 | 0.990 ± 0.444 |
| 8 | 0 | 0.260 ± 0.260 | 1.347 ± 0.818 | 0.674 ± 0.296 |
| 12 | 0 | 1.307 ± 0.393 | 1.354 ± 0.249 | 0.582 ± 0.268 |
| 24 | 0 | 1.094 ± 0.001 | 0 | 1.592 ± 1.231 |

Zeros indicate results to be below the lower limit of detection. Skin Content includes sum of epidermal and dermal content.

### EXAMPLE 4

### Total Skin Content for Isotretinoin Results Across Donors

Percutaneous absorption of isotretinoin with and without benzoyl peroxide into Human Cadaver Skin under light and dark conditions from a single application (Mean±SE (n=2) as Percent of Applied Dose)

TABLE 3

| | | | Ziana ® Gel | Ziana ® Gel |
|---|---|---|---|---|
| | Ziana ® Gel | Ziana ® Gel | without | without |
| | with benzoyl | with benzoyl | benzoyl | benzoyl |
| Exposure | peroxide Gel | peroxide Gel | peroxide Gel | peroxide Gel |
| Duration | Skin Content | Skin Content | Skin Content % | Skin Content % |
| (hours) | % (Light) | % (Dark) | (Light) | (Dark) |
| 2 | 0 | 0 | 0.268 ± 0.268 | 0 |
| 4 | 0 | 0 | 0 | 0 |
| 6 | 0 | 0 | 0 | 0 |
| 8 | 0 | 0 | 0.454 ± 0.454 | 0 |
| 12 | 0 | 0 | 0 | 0 |
| 24 | 0 | 0 | 0 | 0 |

Zeros indicate results to be below the lower limit of detection. Skin Content includes sum of epidermal and dermal content. Table 3 shows that there was no measurable degradation of tretinoin into isotretinoin when the benzoyl peroxide gel was applied. The mass balance accountability is shown below.

TABLE 4

| | Without/With benzoyl | |
|---|---|---|
| | peroxide aqueous gel | Tretinoin recovery over |
| Light/Dark | applied at 2 hours | 24 hours |
| Light | Without | 76.07-81.054% |
| Dark | Without | 95.288-113.148% |
| Light | With | 8.814-14.232% |
| Dark | With | 57.95-65.034% |

This suggests that it is preferable to apply tretinoin aqueous gel with the benzoyl peroxide aqueous gel (whether both tretinoin and benzoyl peroxide are present in the same aqueous gel, or tretinoin in aqueous gel and benzoyl peroxide in another gel and applied sequentially within a short period of time, preferably about 2 hours or less) in the evening.

### EXAMPLE 5

A composition according to the present invention is made in the following manner. Heat and mix paraffin light oil, isostearyl iso stearate, licoleic acid, butyl hydroxyenisol, and phenoxyethanol in the primary vessel until uniform. To the secondary vessel, heat and mix purified water. To this add sodium EDTA and mix until dissolved. Add mixture from the secondary vessel to the main vessel and mix Remove a portion from the main tank and add the Silicone microcapsules and tretinoin. To the main tank add the Pemulen® TR-1 and HPMC and mix sufficiently to hydrate the gelling agents. Add the silicone microcapsules tretinoin mixture to the main tank and mix until homogenous. Add the benzoyl peroxide to the main tank and mix until homogeneous. Add perfume and mix. Cool main tank with mixing to less than 30° C. Adjust pH of the main vessel with sodium hydroxide solution.

### EXAMPLE 6

Acne is treated according to the present invention in the following manner. Wash face gently with mild soap and warm water. Pat the skin dry. Apply a pea-sized amount of tretinoin gel such as the one in Table 5 to fingertips and spread it over the face. Gently smooth it into the skin. Do not get tretinoin gel into the eyes, on mouth, lips, corners of nose, or open wounds.

### EXAMPLE 10

Wash face gently with a mild soap and warm water. Pat the skin dry. Apply a pea-sized amount of Tretinoin/benzoyl peroxide gel to fingertips and spread it over the face. Gently smooth it into the skin. Do not get Tretinoin/benzoyl peroxide gel into the eyes; on mouth, lips, corners of nose, or open wounds.

The present invention may be embodied in other specific forms without departing from its essential characteristics. The described embodiment is to be considered in all respects only as illustrative and not as restrictive. The scope of the present invention is, therefore, indicated by the appended claims rather than by the foregoing description. All changes which come within the meaning and range of the equivalence of the claims are to be embraced within their scope.

What is claimed is:

1. An composition comprising:
water;
benzoyl peroxide;
a retinoid; and
a high molecular weight polymeric gelling agent.

2. The composition of claim 1 wherein the retinoid comprises one or more selected from the group consisting of retinol, retinal, tretinoin, retinoic acid, isotretinoin, alitretinoin and mixtures thereof.

3. The composition of claim 1 wherein the retinoid comprises tretinoin.

4. The composition of claim 3 wherein at least a portion of the tretinoin is solubilized.

5. The composition of claim 3 wherein at least a portion of the tretinoin is crystallized.

6. The composition of claim 5 wherein at least 50% of the tretinoin particles are less than about 10 μm in size and at least 90% of the tretinoin particles are less than 20 μm in size.

7. The composition of claim 1 wherein an aqueous gel is an aqueous phase of an oil in water emulsion.

8. The composition of claim 7, wherein the high molecular weight polymeric gelling agent comprises a polymeric, emulsifying gelling agent.

9. The composition of claim 1, wherein the polymeric gelling agent comprises a polyacrylic acid.

10. The composition of claim 9 wherein the polymeric gelling agent comprises a high molecular weight copolymers of polyacrylic acid.

11. The composition of claim 9 wherein the polymeric gelling agent comprises an emulsifying acrylic acid/C10-C30 alkyl acrylate copolymer.

12. The composition of claim 1, further comprising an antibiotic.

13. The composition of claim 1, further comprising an antioxidant.

14. A method of treating acne comprising:
applying to skin an aqueous gel comprising benzoyl peroxide and a retinoid.

15. The method of claim 14, wherein the retinoid comprises tretinoin.

16. A composition comprising:
tretinoin at about 0.025 weight %;
benzoyl peroxide at about 5 weight %
a high molecular weight polymeric gelling agent; and
water, wherein the composition is substantially free of alcohol.

17. The composition of claim 16, wherein at least about 50% of the tretinoin is crystalline;
at least about 50% of the crystalline tretinoin is less than about 10 μm particle size; and
at least about 90% of the crystalline tretinoin is less than about 20 μm particle size.

18. The composition of claim 16, wherein the polymeric gelling agent comprises a polyacrylic acid gelling agent.

19. The composition of claim 18, wherein the polymeric gelling agent comprises a high molecular weight polyacrylic acid copolymer gelling agent.

20. The composition of claim 18, wherein the polymeric gelling agent comprises an emulsifying acrylic acid/C10-C30 alkyl acrylate copolymer gelling agent.

21. The composition of claim 16, wherein the high molecular weight polymeric gelling agent comprises a polymer belonging to the polyacrylic class of polymers selected from the group consisting of carbomers, carbovinyl polymers, hydrophobically modified carbomers containing polymers, and mixtures thereof.

22. A method of treating acne comprising the step of applying the composition of claims 1-13 to the skin of a patient in need of such treatment.
